(19) United States
(12) Patent Application Publication
(10) Pub. No.: US 2024/0082285 A1
(43) Pub. Date: Mar. 14, 2024

(54) APPLICATION OF CHEBULINIC ACID IN PREPARATION OF ANTI-ROTAVIRUS DRUGS

(71) Applicant: GUANGDONG MEDICAL UNIVERSITY, Dongguan (CN)

(72) Inventors: Lijun Song, Dongguan (CN); Wenchang Zhao, Dongguan (CN); Yupei Qian, Dongguan (CN); Yuxuan Feng, Dongguan (CN); Yujing Zhou, Dongguan (CN)

(21) Appl. No.: 18/510,757

(22) Filed: Nov. 16, 2023

(30) Foreign Application Priority Data
Nov. 16, 2022 (CN) 202211436494.9

(51) Int. Cl. A61K 31/7048; A61P 31/14

(52) U.S. Cl. CPC A61K 31/7048; A61P 31/14

(57) ABSTRACT

The present invention discloses an application of chebulinic acid in preparation of anti-rotavirus drugs, which belongs to the technical field of anti-rotavirus drugs. Chebulinic acid has the effect of resisting rotavirus biosynthesis, has no effect of resistance to rotavirus adsorption and direct killing, and can inhibit the gene expression of VP6 to play an anti-RV role. Network pharmacology analysis indicates that chebulinic acid acts on rotavirus-related genes. Compared with the prior art, the present invention has the beneficial effects that: the present invention relates to an application of chebulinic acid in preparation of anti-rotavirus drugs. In the application of chebulinic acid in preparation of anti-rotavirus drugs, the applicant first finds through research that chebulinic acid has an anti-rotavirus effect, fills the gap of anti-rotavirus drugs, and has profound significance and value, wherein the research shows that the molar concentration of chebulinic acid is 1 μM-10 μM in the anti-rotavirus drugs.

### TECHNICAL FIELD

The present invention relates to the technical field of anti-rotavirus drugs, and more particularly relates to an application of Chebulinic acid in preparation of anti-rotavirus drugs.

### BACKGROUND

Rotavirus (RV), as a member of the Reoviridae family, has an icosahedral structure in appearance and a three-layer concentric protein coat that surrounds the genome composed of 11 dsRNAs, and is responsible for encoding 6 structural proteins and 6 non-structural proteins. RV is the main pathogen that causes infantile diarrhea. The high infectivity and pathogenicity of RV cause great harm to the whole society. At present, there is a lack of specific clinical drugs, and RotaTeq, the only RV vaccine marketed in the United States, is very expensive. Because of the variability and diversity of RV strains, the prevention range for the RV strains is limited. How to prevent and treat RV infection is a major problem.

Chebulinic acid is a phenolic acid structure isolated from Terminalia chebula Retz., acts as a natural M. tuberculosis DNA gyrase inhibitor and can also inhibit the activity of SMAD-3 phosphorylation and H+K+-ATPase. For antivirus, Chebulinic acid has antiviral activity against herpes simplex virus 2 (HSV-2). At the same time, it has been reported in the literature that Chebulinic acid has a certain effect on colon cancer and acute myeloid leukemia.

However, there is no report on the anti-RV effect of Chebulinic acid.

### SUMMARY

In view of this, the present invention provides an application of Chebulinic acid in preparation of anti-rotavirus drugs.

To achieve the above purpose, the present invention adopts the following technical solution:

The application of Chebulinic acid in preparation of anti-rotavirus drugs is provided.

Further, Chebulinic acid can resist rotavirus biosynthesis.

Further, Chebulinic acid can inhibit the gene expression of VP6 to play an anti-rotavirus effect.

An anti-rotavirus drug comprises Chebulinic acid.

Further, the concentration of Chebulinic acid is 1-10 μM.

It can be known from the above technical solution that compared with the prior art, the present invention has the following beneficial effects: in the application of Chebulinic acid in preparation of anti-rotavirus drugs, the applicant first finds through research that Chebulinic acid has an anti-rotavirus effect, fills the gap of anti-rotavirus drugs, and has profound significance and value, wherein the research shows that the molar concentration of Chebulinic acid is 1 μM-10 μM in the anti-rotavirus drugs.

### DESCRIPTION OF DRAWINGS

To more clearly describe the technical solution in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 is a chemical structural formula of Chebulinic acid;

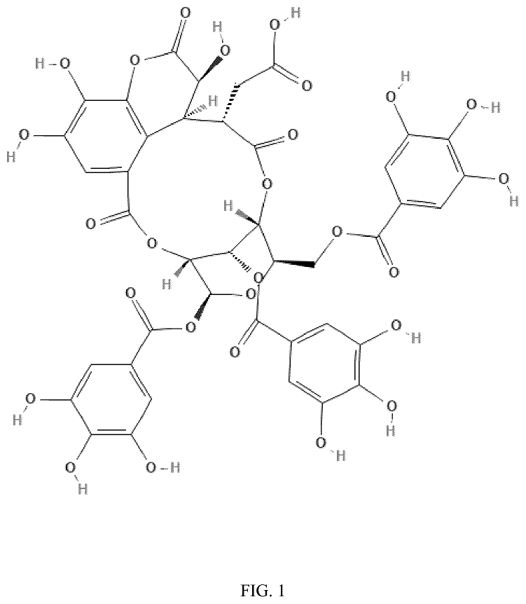

FIG. 2 shows normal MA104 cells in embodiment 1 of the present invention;

FIG. 3 shows MA104 cells after RV infection for 48 h in embodiment 1 of the present invention;

FIG. 4 shows the cell toxicity results of Chebulinic acid in embodiment 1 of the present invention, wherein the normal group represents the untreated group; and compared with the normal group, *** p<0.001 and ****p<0.0001;

FIG. 5 shows the effect of Chebulinic acid against RV adsorption in embodiment 1 of the present invention, wherein compared with the ribavirin group, ****p<0.0001;

FIG. 6 shows the direct killing effect of Chebulinic acid on RV in embodiment 1 of the present invention, wherein compared with the ribavirin group, * p<0.05 and ** p<0.01;

FIG. 7 shows the effect of Chebulinic acid against RV biosynthesis in embodiment 1 of the present invention;

FIG. 8 shows the effect of Chebulinic acid for inhibiting gene expression of structural protein VP6 in RV-infected MA104 cells in embodiment 1 of the present invention, wherein compared with the virus group, * * ** p<0.0001;

FIG. 9 is a rotavirus PPI network diagram in embodiment 2 of the present invention;

FIG. 10 is a “drug-target” network diagram in embodiment 2 of the present invention;

FIG. 11 is a Venn diagram of RV and Chebulinic acid in embodiment 2 of the present invention;

FIG. 12 is an intersection gene network diagram of RV and Chebulinic acid in embodiment 2 of the present invention.

### DETAILED DESCRIPTION

The technical solutions in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

Materials and instruments used in the embodiments are as follows:

TABLE 1
Experimental Cell Lines and Reagents

| Material Name | Manufacturer |
|---|---|
| Chebulinic acid, batch number | Chengdu Ruifensi Biotechnology Co., |
| RFS-L10402007031 | Ltd. |
| MA104 cell line | Sun Yat-sen University Cell Library |
| MA104-2 cell line | Wuhan University Cell Library |
| Wa strain RV | Third Military Medical University, |
| | Immunology Institute |
| 1 × PBS phosphate buffer | Jiangsu Beyotime Biotechnology |
| | Institute |
| Cellular dimethyl sulfoxide | American GIBCO company |
| Fetal bovine serum | American GIBCO company |
| High-glucose DMEM culture | American GIBCO company |
| solution | |
| 0.25% trypsin digestion solution | Beijing Solarbio Science & Technology |
| | Co., Ltd. |
| 0.25% EDTA-containing trypsin | Beijing Solarbio Science & Technology |
| digestion solution | Co., Ltd. |
| Penicillin-streptomycin solution | Beijing Solarbio Science & Technology |
| (double antibody) | Co., Ltd. |
| Confocal dish | Beijing Labgic Technology Co., Ltd. |
| Pipette tip | Shanghai Kirgen Biotechnology Co., |
| | Ltd. |
| Cell culture flask | American Corning-Coster company |
| Cell culture dish | American Corning-Coster company |
| Culture plate | American Corning-Coster company |

TABLE 2
Main Instruments

| Instrument Name | Manufacturer |
|---|---|
| ordinary optical microscope | Japanese OLYMPUS company |
| Inverted fluorescent microscope | Japanese NIKON company |
| Micropipettor | Germany Eppendorf company |
| −20° C. refrigerator | Midea company |
| −80° C. ultra low temperature | Thermo Scientific company |
| refrigerator | |
| CO₂ incubator | Thermo Scientific company |
| Autoclave | Japanese Sanyo company |
| Super clean bench | Suzhou Airtech company |
| Electric-heated thermostatic water | Shanghai Yiheng Science and |
| bath | Technology Co., Ltd. |
| Multimode reader | Gene Company Linited company |
| Nucleic acid protein analyzer | Germany Eppendorf company |
| Living cell imager | Thermo Scientific company |
| Illumination incubator | Shanghai Yiheng Science and |
| | Technology Co., Ltd. |
| Thermal cycler | T100 Bio-Rad company |
| PCR instrument | Thermo Scientific company |
| High-speed freezing centrifuge | Germany eppendorf company |
| Vortex shaker | Ika Instrument and Equipment |
| | Co., Ltd. |
| Liquid nitrogen container | Chart Biology Co., Ltd. |

Preparation methods for main reagents are as follows:

Chebulinic acid mother solution: 5.7 mg of Chebulinic acid standard product with purity≥98% and batch number RFS-L10402007031 is weighed; 1 mL of cell-grade DMSO is added in a super clean bench for dissolving; and the drug solution is filtered through a 0.22 μM filter membrane with a disposable sterile needle to remove bacteria; and 10 mM of mother solution is prepared.

DMEM (containing 10% fetal bovine serum and 1% double antibody): 50 mL of fetal bovine serum and 5 mL of double antibody are successively added into a 400 mL high-glucose DMEM in the super clean bench, thoroughly mixed evenly, and then packaged into labeled 50 mL centrifuge tubes; and the centrifuge tubes are sealed, and stored at 4° C.

10 μg/mL EDTA-free pancreatin: 0.25% EDTA-free trypsin digestion solution and fetal bovine serum-free DMEM are diluted to 10 μg/mL at a 1:250 volume ratio.

RV growth maintenance solution: 10 μg/mL EDTA-free pancreatin and fetal bovine serum-free DMEM are diluted to 1 μg/mL at a 1:10 volume ratio.

Analysis of Statistical Data:

All experiments are repeated for three times. The experimental data are expressed by mean±standard deviation (x±s). SPSS software is used for statistical data. The t-test is used for comparison between two groups, and one-way analysis of variance is used for comparison of the means among multiple groups. P<0.05 indicates that the difference has statistical significance. The obtained data results are expressed in the form of mean±standard deviation. Statistical analysis software Graphpad prism 8.01 is used for Mann-Whitney statistics in data analysis.

The Wa strain RV is selected in the embodiment because of the better susceptibility to people.

### Embodiment 1

(1) RV infects MA104 cells

The virus is melted in a water bath at 37° C. and pretreated with trypsin for 30 min.

The virus solution is added to MA104 cells that grow to 80-90%. No serum is added to a cell culture medium used for RV infection. The MA104 cells after virus infection generate cytopathic effect (CPE). When the degree of CPE reaches 75%, the MA104 cells are frozen in a refrigerator at −20° C. After repeated the freezing and melting process for 3 times, the MA104 cells are centrifuged at low temperature and the supernatant is collected, which is the virus solution. The above process is repeated and the RV is amplified.

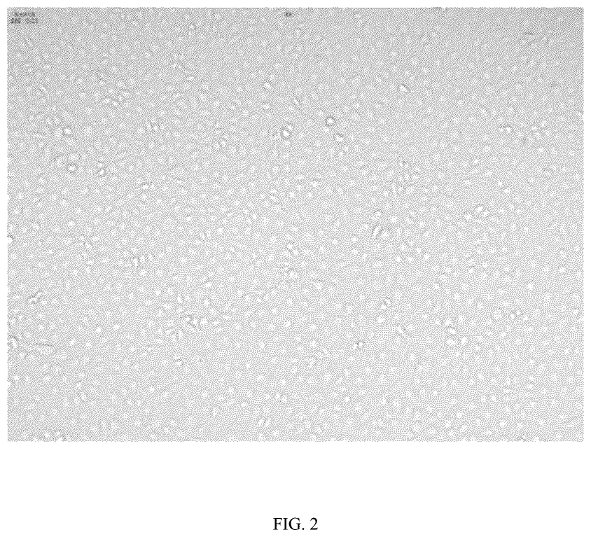

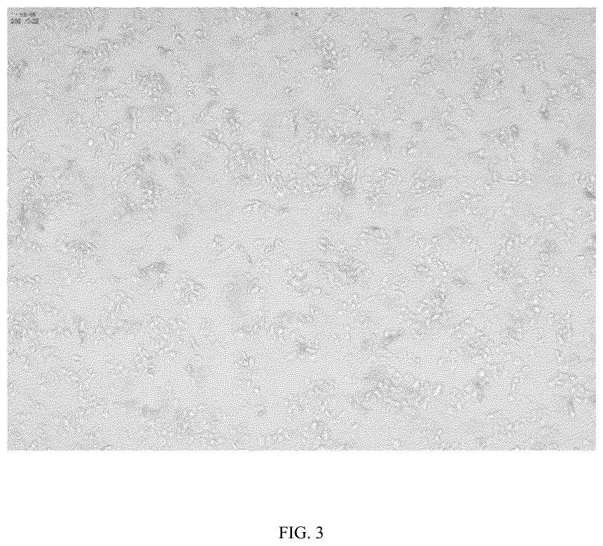

Normal MA104 cells and the MA104 cells after 48 hours of RV infection are shown in FIGS. 2 and 3. It can be seen that the normal MA104 cells are triangular or fusiform, and the cell outlines are clear. The MA104 cells after RV infection have obvious CPE; the cell boundaries become blurred, and the distance between the cells is increased. Finally, the cells completely come off and float.

(2) The toxicity of Chebulinic acid to the MA104 cells is detected by CCK8 method

According to the common drug concentration range of Chebulinic acid, the cytotoxicity pre-experiment is performed between 1 μM and 25 μM in this experiment. The MA104 cells at the logarithmic growth stage are observed under a microscope. The cellular morphology is uniform and full with clear edges, and when the number reaches 80%, digestion, centrifugation and re-suspension are conducted. After further dilution of the suspension, 10 L is taken on a blood cell counting plate to count and calculate the required cell volume. Then, the cells are laid in a 96-well plate, 100 L of cell suspension is added to each well, and the cell density is 8×104 cells/mL. When the cells adhere to the wall to form a single layer, the drug group is administered, and the normal group is added with an equal volume of DMEM (without serum). After incubation for 48 h, the cytotoxicity of Chebulinic acid is detected by CCK8 reagent. 1/10 volume of CCK8 solution is added to each well and incubated in an incubator. After 90 min, the absorbance is detected and recorded under the condition of oscillation for 10 s and at 450 nm wavelength. The formula for the relative survival rate of the cells is:

{(A_{xpenmentai group}−A_{blank})/(A_{control group}−A_{blaimk})×100%}.

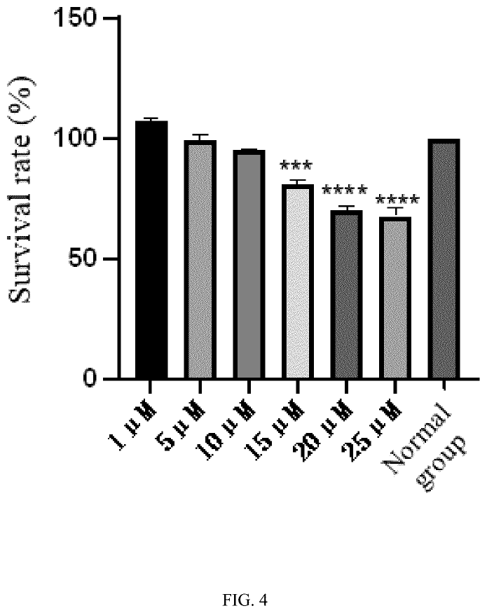

The results are shown in FIG. 4. The drug concentration of Chebulinic acid is between 1 μM and 10 μM, which has no drug toxicity to the cells, and the average survival rate is above 95%. Compared with the N group, there is no significant difference in cell viability between the drug group and the N group when Chebulinic acid is 1-10 μM. Therefore, the selected experimental drug concentration of Chebulinic acid is 1-10 μM.

(3) Three anti-RV effects of Chebulinic acid and effects on gene expression of structural protein VP6 in RV-infected MA104 cells are detected by CCK8 method

To research whether Chebulinic acid has the effect of anti-RV infection in vitro, relevant experiments are conducted on MA104 cell models from three effects of Chebulinic acid against RV adsorption, direct inactivation and biosynthesis, and the effects on gene expression of structural protein VP6 in RV-infected MA104 cells.

\textcircled{1} (Effect of Chebulinic Acid Against RV Adsorption

The 96-well culture plate of the MA104 cells that grow to the single layer is added with the drug solution. Each drug solution is repeated with 6 wells, and each well contains 100 μL. An equal volume of ribavirin is added to the positive control group, and only an equal volume of DMEM culture solution (without serum) is added to the normal cell control group and the viral control group. Incubation is conducted at 37° C. and 5% CO₂ for 2 h. The drug solution is sucked out. Except the normal cell control group, 100 TCID50 virus is added (the virus acts with 10 μg/mL pancreatin at 37° C. for 30 min), and each well contains 100 μL. Incubation is conducted at 37° C. and 5% CO₂ for 2 h. The virus is sucked out, the cell maintenance solution is added, and each well contains 200 μL. Incubation is conducted at 37° C. and 5% CO₂ for continuous observation. After culture for 48 h, CCK-8 kit is used for detection. 1/10 volume of CCK-8 solution is added to each well, and incubated in the incubator. After 90 min, the absorbance is detected and recorded at 450 nm wavelength. The experiment is repeated for 3 times. The formula of the viral inhibition rate is:

{(A_{drug group}−A_{virus group})/(A_{normal group}−A_{virus group})×100%}.

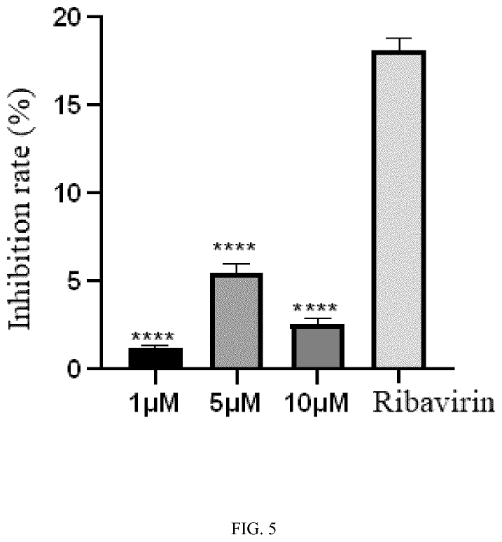

The results are shown in FIG. 5. Compared with the ribavirin group, the anti-RV inhibition rates of the Chebulinic acid group are not significantly increased at 1 μM, 5 μM and 10 μM, with statistical difference, and the highest inhibition rate is only about 5%, which indicates that Chebulinic acid has no obvious anti-RV adsorption effect.

\textcircled{2} (Direct Killing Effect of Chebulinic Acid on RV

The drug is mixed with 100 TCID50 virus solution (the virus acts with 10 μg/mL pancreatin for 30 min) at an equal volume for 2 h. After the cells are washed twice with PBS, the cells are added to the 96-well culture plate of the MA104 cells that grow to the single layer. In the positive control group, the same operation as above is conducted for ribavirin and RV, and only an equal volume of DMEM (without serum) is added to the normal cell control group and the viral control group. Incubation is conducted at 37° C. and 5% CO₂ for 2 h. Then, the mixed solution is sucked out, the cell maintenance solution is added, and each well contains 100 μL. Incubation is conducted at 37° C. and 5% CO₂ for continuous observation. After culture for 48 h, CCK-8 kit is used for detection. 1/10 volume of CCK-8 solution is added to each well, and incubated in the incubator. After 90 min, the absorbance is detected and recorded at 450 nm wavelength. The viral inhibition rate of the drug is calculated, and the experiment is repeated for 3 times. The formula of the viral inhibition rate is:

{(A_{drug group}−A_{virus group})/(A_{normal group}−A_{virus group})×100%}.

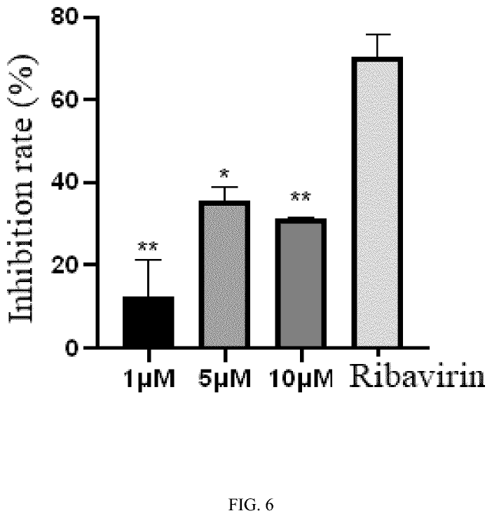

The results are shown in FIG. 6. Compared with the ribavirin group, the inhibition rate for RV in each Chebulinic acid group is lower, and there are significant differences in statistical comparison, which indicates that Chebulinic acid has no obvious direct killing effect on RV.

\textcircled{3} Effect of Chebulinic Acid Against RV Synthesis

100 TCID50 virus solution (the virus acts with 10 μg/mL pancreatin for 30 min) is added to the 96-well culture plate of the MA104-2 cells that grow to the single layer, and each well contains 100 μL. The cells have been washed twice with PBS. Normal cell control is set, and added with an equal volume of DMEM. Incubation is conducted at 37° C. and 5% CO₂ for 2 h. Then, the virus solution is sucked out and added into different concentrations of drug solution and ribavirin respectively, and each well contains 100 μL. Virus control is set. Only the cell maintenance solution is added, and each well contains 100 μL. Incubation is conducted at 37° C. and 5% CO₂ for continuous observation. After continuous culture for 48 h, CCK-8 kit is used for detection. 1/10 volume of CCK-8 solution is added to each well, and incubated in the incubator. After 90 min, the absorbance is detected and recorded at 450 nm wavelength. The viral inhibition rate of the drug is calculated, and the experiment is repeated for 3 times. The formula of the viral inhibition rate is:

{(A_{drug group}−A_{virus group})/(A_{normal group}−A_{virus group})×100%}.

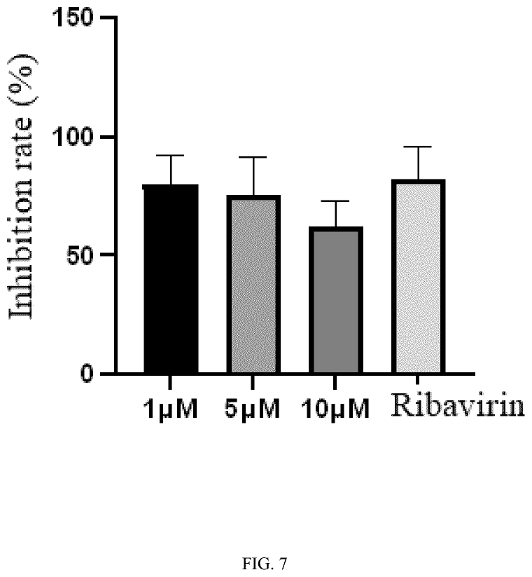

The results are shown in FIG. 7. Compared with the ribavirin group, the anti-RV inhibition rates of Chebulinic acid reach 80.01%, 75.26% and 61.86% at 1 μM, 5 μM and 10 μM. There is no significant difference in statistical comparison, which indicates that Chebulinic acid and ribavirin have an obvious effect of synthesis inhibition on RV and indicates that Chebulinic acid has an obvious effect of synthesis inhibition on RV.

\textcircled{4} Effects of Chebulinic Acid on Gene Expression of Structural Protein VP6 in RV-Infected MA104 Cells

The Experimental Method is as Follows:
(1) Extraction and quantification of total RNA;
(2) Primer sequence design;
(3) Reverse transcription reaction;
(4) SYBR Green I fluorescent labeling is used for real-time quantitative PCR detection of expression of VP6 gene.

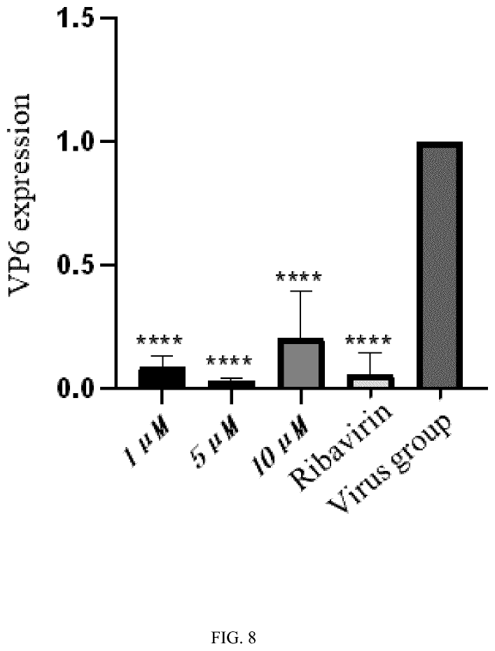

The results are shown in FIG. 8. Compared with the RV group, VP6 expression in the Chebulinic acid group is significantly decreased at concentrations of 1 μM, 5 M and 10 μM, with statistical difference, wherein Chebulinic acid inhibits VP6 expression most significantly at 5 μM. It indicates that Chebulinic acid plays an anti-RV effect by inhibiting VP6 gene expression.

To sum up, Chebulinic acid has the effect of resisting RV biosynthesis, has no effect of resistance to RV adsorption and direct killing, and can inhibit the gene expression of VP6 to play an anti-RV role.

### Embodiment 2 Network Pharmacology Research on Chebulinic Acid Against Rotavirus

(1) Construction of PPI Network Diagram of RV Targets

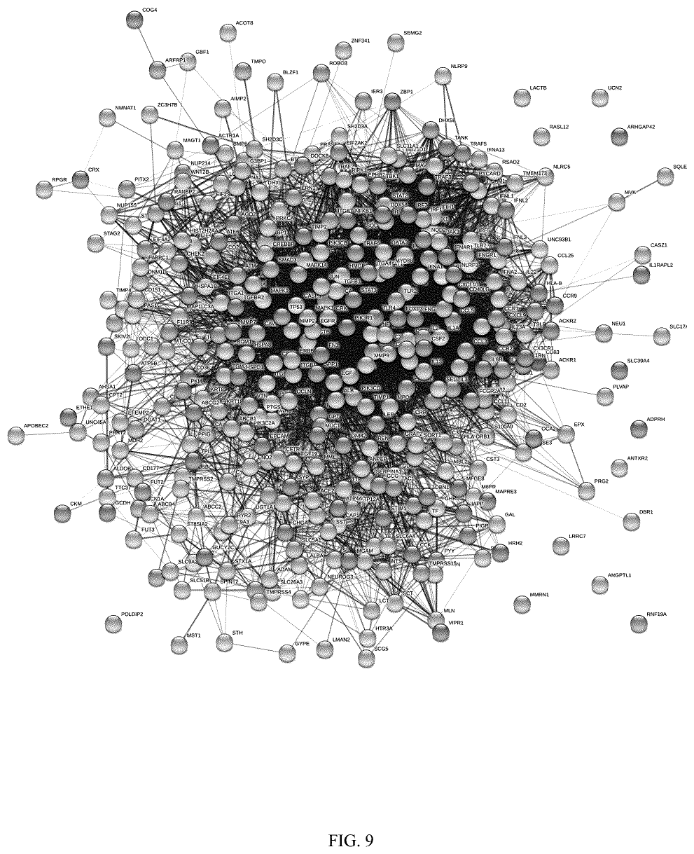

RV targets are found in Genecards, OMIM and Disgenet databases, and the collected targets are used to construct a rotavirus PPI network diagram through the STRING website (FIG. 9).

(2) Construction of Chebulinic Acid “Drug-Target” Network Diagram

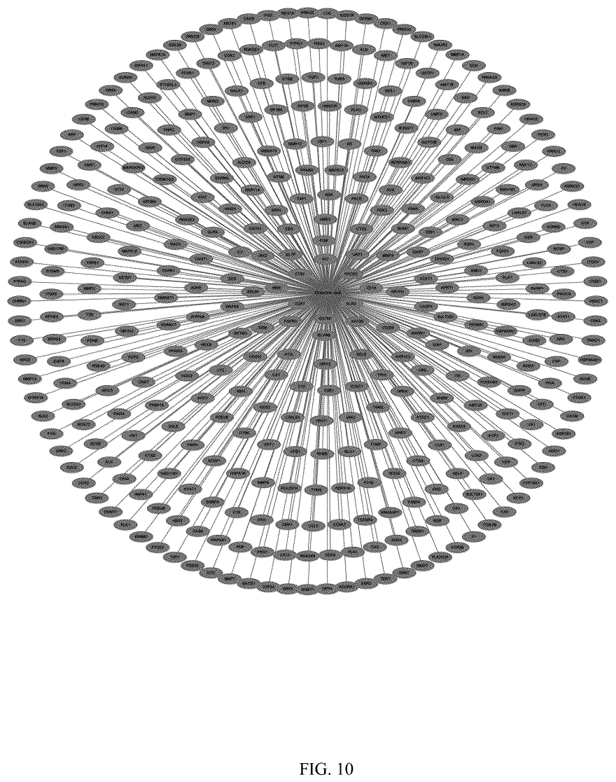

Chebulinic acid-related genes are collected through Swisstargetprediction and Pharmmapper databases, and the collected targets are used to draw a “drug-target” network diagram through Cytoscape software (FIG. 10).

(3) Construction of Intersection Gene Network Diagram by Venn Diagram

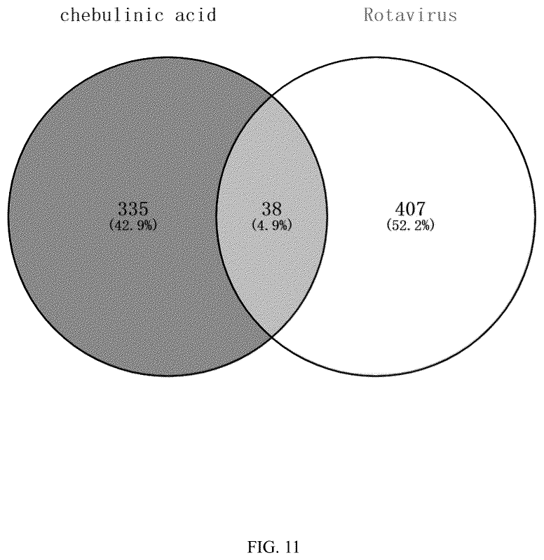

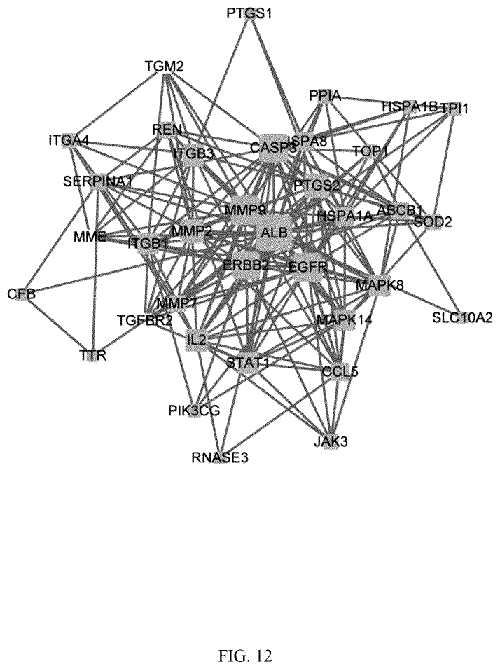

The related intersection genes are obtained by the RV targets and the Chebulinic acid targets through the Venn diagram (FIG. 11), and the obtained intersection genes are used for drawing the intersection gene network diagram through Cytoscape software (FIG. 12).

Network pharmacology analysis indicates that Chebulinic acid acts on the RV-related genes.

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention.

Therefore, the present invention will not be limited to these embodiments shown herein, but will conform to the widest scope consistent with the principle and novel features disclosed herein.

What is claimed is:

1. An application of chebulinic acid in preparation of anti-rotavirus drugs.

2. The application according to claim 1, wherein chebulinic acid is used for anti-rotavirus biosynthesis.

3. The application according to claim 1, wherein chebulinic acid is used for inhibiting the gene expression of VP6 to play an anti-rotavirus effect.
